(19) United States
(12) Patent Application Publication
(10) Pub. No.: US 2024/0102057 A1
(43) Pub. Date: Mar. 28, 2024

(54) METHODS AND COMPOSITIONS USEFUL FOR THE PRODUCTION OF 4-VINYLPHENOL

(71) Applicants: THE REGENTS OF THE UNIVERSITY OF CALIFORNIA, Oakland, CA (US); NATIONAL TECHNOLOGY & ENGINEERING SOLUTIONS OF SANDIA, LLC, Albuquerque, NM (US)

(72) Inventors: Alberto RODRIGUEZ, Concord, CA (US); Jamie A. MEADOWS, East Greenwich, RI (US); Ning SUN, Fremont, CA (US); Blake A. SIMMONS, San Francisco, CA (US); John M. GLADDEN, Alameda, CA (US)

(21) Appl. No.: 18/466,660

(22) Filed: Sep. 13, 2023

Related U.S. Application Data
(60) Provisional application No. 63/375,568, filed on Sep. 14, 2022.

(51) Int. Cl. C12P 7/22; C12N 9/18

(52) U.S. Cl. CPC C12P 7/22; C12N 9/18; C12Y 301/01093

(57) ABSTRACT

The present invention provides for a method for producing a 4-vinylphenol (4VP) and/or 4-vinylguaiacol (4VG), the method comprising: (a) providing a host cell capable of expressing a polypeptide having a phenolic acid decarboxylase (PAD) enzymatic activity wherein the polypeptide is capable of converting p-coumaric (CA) and/or ferulic acid (FA) into 4-vinylphenol (4VP) and/or 4-vinylguaiacol (4VG), respectively; and (b) culturing the host cell in a culture medium to express the polypeptide such that the polypeptide converts CA and/or FA into 4VP and/or 4VG, respectively; wherein the culture medium comprises an organic overlay or phase.

### CROSS-REFERENCE TO RELATED APPLICATIONS

This application claims priority to U.S. Provisional Patent Application Ser. No. 63/375,568, filed on Sep. 14, 2022, which is hereby incorporated by reference.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### REFERENCE TO SEQUENCE LISTING

The instant application contains a Sequence Listing which has been submitted electronically in XML format and is hereby incorporated by reference in its entirety. Said XML copy, created on Sep. 13, 2023, is named “2021-122-02_SequenceListing.xml” and is 7 kilobytes in size.

### FIELD OF THE INVENTION

The present invention is in the field of production of producing 4-vinylphenol.

### BACKGROUND OF THE INVENTION

The discovery of sustainable and affordable routes for the production of commodity and specialty biochemicals from renewable sources is essential to reduce the global demand for petroleum and other fossil materials. Lignocellulosic biomass comprises one of the largest reservoirs of renewable carbon in the planet and represents an attractive source of chemical building blocks that can be refined and upgraded into biofuels and bioproducts ([14, 15]). Among the main constituents of lignocellulosic biomass, cellulose and hemicellulose are polymeric carbohydrates that can be deconstructed to monomeric sugars and converted to biofuels and other valuable bioproducts [4]. The third main component is lignin, an aromatic heteropolymer with high chemical complexity that makes it more resistant to deconstruction and conversion [30]. Considering that this complexity also opens the possibility to valorize a wide array of aromatic compounds, efforts to depolymerize lignin and chemically or biologically upgrade lignin monomers have steadily increased over the last decade [2, 23, 29]. For example, hydroxycinnamic acids such as p-coumaric (CA) and ferulic (FA) acids are precursors of lignin biosynthesis in plants and are chemically linked to lignin grassy biomass at considerable levels (5-15% wt.) [20, 26]. These types of compounds could represent a viable option for valorization of aromatic compounds derived from lignocellulosic feedstocks because they can be extracted through mild thermochemical treatments, are fully metabolized by certain fungal and bacterial species, and their relative amounts could be increased through bioengineering of crops [14, 15, 21, 24]. In particular, CA and FA can be decarboxylated to their vinylphenol derivatives, which hold a high market value due to their applications as biopolymer precursors, perfumes, and food additives.

Several groups have explored the feasibility of using enzymatic approaches to convert CA to 4-vinylphenol (4VP) or FA to 4-vinylguaiacol (4VG) through a single reaction catalyzed by a phenolic acid decarboxylase (PAD) enzyme [5, 8, 10, 16, 16, 17, 17]. Over 100 bacterial species are predicted to contain genes coding for PAD enzymes but the best characterized enzymes to date belong to members of the Bacillus and Lactobacillus genera [18]. In addition to using organisms with the natural ability to decarboxylate CA or FA, several PAD enzymes have been heterologously expressed in E. coli and either purified for in vitro reactions or the recombinant strains used in whole-cell conversion methods [1, 10, 25]. However, 4VP has proven to be significantly more toxic to E. coli than CA and there are reports of product inhibition in PAD enzymes [12]. Some strategies that have been employed to mitigate this problem include the use of biphasic systems with an organic overlay, extractive fermentation, and fed-batch approaches. In particular, hexane and octanol are two organic solvents that have proven to be effective at extracting 4VP from the aqueous phase but are also known to cause significant toxicity to E. coli and inhibition of PAD enzyme activity [10, 16, 17, 25].

### SUMMARY OF THE INVENTION

The present invention provides for a method for producing a 4-vinylphenol (4VP) and/or 4-vinylguaiacol (4VG), the method comprising: (a) providing a host cell capable of expressing a polypeptide having a phenolic acid decarboxylase (PAD) enzymatic activity wherein the polypeptide is capable of converting p-coumaric (CA) and/or ferulic acid (FA) into 4-vinylphenol (4VP) and/or 4-vinylguaiacol (4VG), respectively; and (b) culturing the host cell in a culture medium to express the polypeptide such that the polypeptide converts CA and/or FA into 4VP and/or 4VG, respectively; wherein the culture medium comprises an organic overlay or phase.

In some embodiments, the method further comprises: (c) optionally recovering the 4VP and/or 4VG. In some embodiments, the recovering step comprises distilling, such as low pressure or vacuum distilling, to separate the 4VP and/or 4VG from the saturated or unsaturated fatty alcohol. In some embodiments, the difference between the boiling point of 4VP or 4VG and the boiling point of the saturated or unsaturated fatty alcohol is equal to or more than about 10° C., 20° C., 30° C., 40° C., or 50° C.

In some embodiments, the method is a method for producing a 4-vinylphenol (4VP), the method comprising: (a) providing a host cell capable of a polypeptide having a phenolic acid decarboxylase (PAD) enzymatic activity wherein the polypeptide is capable of converting p-coumaric (CA) into 4-vinylphenol (4VP); and (b) culturing the host cell in a culture medium to express the polypeptide such that the polypeptide converts CA into 4VP, respectively; wherein the culture medium comprises an organic overlay or phase.

In some embodiments, the method is a method for producing a 4-vinylguaiacol (4VG), the method comprising: (a) providing a host cell capable of a polypeptide having a phenolic acid decarboxylase (PAD) enzymatic activity wherein the polypeptide is capable of converting ferulic acid (FA) into 4-vinylguaiacol (4VG); and (b) culturing the host cell in a culture medium to express the polypeptide such that the polypeptide converts FA into 4VG; wherein the culture medium comprises an organic overlay or phase.

The present invention provides for genetically modified host cells, such as an engineered E. coli strain, that can produce esters of isoprenoid derived alcohols. In some embodiments, the genetically modified host cells, such as an engineered E. coli strain, are modified via episomal expression of a heterologous mevalonate pathway with the isopentenyl diphosphate (IPP) bypass and a promiscuous phosphatase to generate isoprenol. In some embodiments, further expression of an alcohol acyltransferase, ATF1, catalyzes the acetylation of isoprenol and acetyl-CoA to generate isoprenyl acetate, a high value chemical used as a fuel, fragrance, or food additive with significant market potential. High titer production has been demonstrated in laboratory scale batch and fed-batch 2 L reactors on glucose. This strain has demonstrated the highest yet reported titer of isoprenyl acetate with an approximately 100-fold improvement over current means. In some embodiments, the platform can be expanded to include different alcohols, such as the isomer prenol for prenyl acetate production, and to include other carboxylic acid-CoA thioesters including, but not limited to, lactyl-CoA, propionyl-CoA, and dimethylacrylyl-CoA. In some embodiments, the platform could also be modified for growth on other carbon substrates like pretreated lignocellulosic hydrolysate.

In some embodiments, the genetically modified host cell has the expression of pta, poxB, and/or ackA reduced, knocked out, or deleted or knocked out, or a nucleic acid encoding the pta, poxB, and/or ackA operatively linked to an inducible promoter. In some embodiments, the genetically modified host cell has its endogenous expression of pta, poxB, and/or ackA reduced, knocked out, or deleted or knocked out, or a nucleic acid encoding the pta, poxB, and/or ackA operatively linked to an inducible promoter or a promoter with a transcriptional strength lower than the native promoters thereof.

Typically, deletion of pta, poxB, and ackA result in near complete loss of acetate production, which would ideally drive an accumulation of acetyl-CoA. Increased expression of Acetyl-CoA synthetases (ACS) increases conversion of acetate into acetyl-CoA substrate. In some embodiments, the host cell comprises an acetyl-CoA synthetases (ACS), or homologous enzyme thereof. In some embodiments, the host cell is genetically modified by the introduction of an ACS, or homologous enzyme thereof.

In some embodiments, the polypeptide comprises an amino acid sequence having at least about 70%, 75%, 80%, 85%, 90%, 95%, or 99% amino acid sequence identity with SEQ ID NO:l. In some embodiments, the polypeptide comprises an amino acid sequence of a PAD found in nature, or an engineered enzyme thereof

In some embodiments, the polypeptide comprises one or more of the following conserved amino acid residues: binding site (Mn²⁺) at amino acid position 160, prenyl-FMN binding region at amino acid residue positions 160-165, prenyl-FMN binding region at amino acid residue positions 181-182, binding site (Mn²⁺) at amino acid position 182, binding site (Mn²⁺) at amino acid position 224, and/or proton donor active site at amino acid position 273.

In some embodiments, the polypeptide comprises one or more of the following conserved amino acid residues: N at amino acid position 160, NVGIYR (SEQ ID NO:2) at amino acid residue positions 160-165, QH at amino acid residue positions 181-182, H at amino acid position 182, E at amino acid position 224, and/or E at amino acid position 273.

In some embodiments, the genetically modified host cell is a host cell or microorganism, or comprises one or more aspects thereof, disclosed by U.S. Pat. Nos. 7,985,567; 9,631,210; 10,273,506; and, 10,814,724; and, PCT International Patent Application Nos. PCT/US2008/068831 and PCT/US2012/055165 (herein incorporated by reference).

In some embodiments, the culturing of the host cell comprises employing an organic overlay or phase on the culture. In some embodiments, the organic overlay or phase comprises a saturated or unsaturated fatty alcohol, such as undecanol, as a mechanism for sequestering the 4VP or 4VG product from the aqueous solution. Although the overlay or phase facilitates chemical extraction, its application may not be feasible at scale. Likewise, further analysis must be conducted to assess the relative feasibility of downstream separation technologies for acceptable isolation of pure product. In some embodiments, the saturated fatty alcohol has the following chemical structure: R″-OH, wherein R″ is a C₁ to C₂₀ alkyl. In some embodiments, the R″ is an alkyl with 1, 2, 3, 4, 5, 6, 7, 8, 9, 10, 11, 12, 13, 14, 15, 16, 17, 18, 19, or 20 carbon atoms, or having a range of any two preceding values thereof. In some embodiments, the unsaturated fatty alcohol has the following chemical structure: α-CR═CR′-β-OH, wherein α and β are each independently a C₁ to C₂₀ alkyl, and R, and R′ are each independently H or a C₁ to C₂₀ alkyl, wherein 1, 2, 3, or 4 of α, β, R, and R′ comprises one or more hydroxyl groups. In some embodiments, α and β are each independently a C₅ to C₂₀ alkyl. In some embodiments, R and R′ are each independently H or a C₅ to C₂₀ alkyl. In some embodiments, the unsaturated fatty alcohol is an unsaturated fatty alcohol described in Egan et al. “Properties and uses of some unsaturated fatty alcohols and their derivatives,” J. Amer. Oil Chem. Soc., 61(2): 324-329, 1984 (herein incorporated by reference). Oleyel alcohol has the following chemical structure:

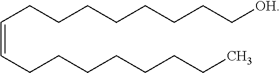 [SMILES: CCCCCCCC/C=C\CCCCCCCCO]

In some embodiments, the organic overlay or phase comprises an alkanol, such as octanol, nonanol, octanol, decanol, undecanol, dodecanol, or tridecanol, or a mixture thereof. In some embodiments, the saturated or unsaturated fatty alcohol, such as the alkanol, is a liquid at about room temperature, or about 20° C., 25° C., 30° C., 35° C., or 40° C.

In some embodiments, the saturated or unsaturated fatty alcohol is not toxic to the host cell, such as E. coli, and/or the saturated or unsaturated fatty alcohol does not inhibit the enzymatic activity of PAD. In some embodiments, the saturated or unsaturated fatty alcohol has a toxicity to the host cell, such as E. coli, equal to or less than that of undecanol, and/or the saturated or unsaturated fatty alcohol inhibits the enzymatic activity of PAD equal to or less than that of undecanol. In some embodiments, the saturated or unsaturated fatty alcohol has a toxicity to the host cell, such as E. coli, equal to or more than that of undecanol, and/or the saturated or unsaturated fatty alcohol inhibits the enzymatic activity of PAD equal to or more than that of undecanol.

In some embodiments, the method results in the 4VP and/or 4VG extracted and concentrated in the organic phase reaches a titer equal to or more than about 100 g/L, 110 g/L, 120 g/L, 130 g/L, 140 g/L, 150 g/L, 160 g/L, 170 g/L, 180 g/L, or 190 g/L, ora range between any two preceding values.

In some embodiments, the method results in 10%, 20%, 30%, 40%, 50%, 60%, 70%, 80%, or 90%, or a range between any two preceding values, of the CA and/or FA in the media are converted into 4VP and/or 4VG, respectively. In some embodiments, the method results in equal to or more than about 10%, 20%, 30%, 40%, 50%, 60%, 70%, 80%, or 90%, or a range between any two preceding values, of the CA and/or FA in the media are converted into 4VP and/or 4VG, respectively.

In some embodiments, the CA and/or FA are derived or obtained from a biomass, such as lignocellulose or any lignin-containing substrate. In some embodiments, the lignocellulose or lignin-containing substrate is derived or obtained from a pretreatment, such as a dilute acid pretreatment and optionally comprising an enzymatic saccharification of the biomass. In some embodiments, the biomass is from a solid waste stream, such as one generated from a lignocellulosic biorefinery. To extract CA and/or FA, the biomass is first solubilized under a condition that both promotes the release of CA and/or FA and generate a biocompatible liquor (i.e. having a pH and salt concentrations within the physiological range of the microbes and low amounts of toxic phenolic compounds). A CA yield close to about 10 wt % of the total biomass content can be obtained with a base-catalyzed depolymerization method. In some embodiments, a lignin liquor is prepared from the water-insoluble residue of a biomass (such as containing about 55 wt % lignin), obtained after acid pretreatment and saccharification reactions. In some embodiments, a relatively high solid load of about 25% (w/v) can be used in the depolymerization reaction to maximize the CA and/FA concentration in the solution. The resulting CA- and/or FA-rich liquor can be pH-adjusted to about 7.5 and a CA and/or FA concentration of about 8 g/L.

This invention describes a process for cultivating a host cell, such a bacterium, that express a phenolic acid decarboxylase enzyme for conversion of CA to 4VP and/or FA to 4VG. By coupling high cell density cultivations with the use of a saturated or unsaturated fatty alcohol, such as undecanol, overlay or phase, equal to or more than about 90% of the coumaric acid in the media can be converted to 4-vinylphenol and the 4VP and/or 4VG extracted and concentrated in the organic phase to reach titers as high as about 187 g/L.

In some embodiments, the host cell comprises a nucleic acid encoding the polypeptide operatively linked to a promoter into the host cell. In some embodiments, method further comprises, prior to the providing step, introducing a nucleic acid encoding the polypeptide operatively linked to a promoter into the host cell. The promoter is capable of initiating transcription of the mRNA encoding the polypeptide in the host cell.

In some embodiments, the nucleotide sequence of the polypeptide is codon optimized for the host cell. The gene sequence coding for a phenolic acid decarboxylase from B. amyloliqufaciens was codon optimized for B. subtilis, E. coli and C. glutamicum and cloned by GenScript (Piscataway, NJ).

This invention relates to the valorization of lignin, one of the main components of lignocellulosic biomass, by converting the lignin-derived compound p-coumaric acid (CA) to the bioplastics precursor 4-vinylphenol (4VP). Here we disclose an approach to release CA from corn stover lignin and to biologically convert it to 4VP.

In some embodiments, the host cell is genetically modified to delete a gene encoding an acyl:CoA ligase enzyme (phdA-) was also constructed and transformed with the same plasmid.

The use of enzymatic methods to convert CA to 4VP is documented in the literature and based on the heterologous expression of PAD enzymes from Bacillus and Lactobacillus species in E. coli. These enzymes have either been purified to perform catalysis in vitro or the recombinant strains employed in whole-cell conversion procedures that involve the use of biphasic solvent systems, extractive fermentation in bioreactors or fed-batch approaches. However, only few solvents have been experimentally tested for extraction of 4VP from the aqueous phase and, although octanol and hexane can achieve this purpose, they are notoriously toxic to E. coli and are known to be inhibitors of PAD enzyme activity.

In some embodiments, the method is continuous and further comprises continuous or multiple introducing or adding of fresh culture medium, separating some or a portion of the organic overlay or phase containing the 4VP and/or 4VG from the culture medium, and continuous or multiple adding or introducing organic overlay or phase. In some embodiments, the 4VP and/or 4VG are separated from the organic overlay or phase, and optionally the organic overlay or phase is added or introduced to the culture medium comprising the host cell.

In some embodiments, the method results in the continuous extraction of 4VP and/or 4VG from the liquid phase with an organic overlay or phase coupled to the use of high initial cell densities led to higher product concentrations, with most of the product being recovered in the organic phase. In some embodiments, the use of undecanol increased the conversion capabilities of all organisms, especially when using higher CA concentrations in the media. In some embodiments, the recombinant C. glutamicum phdA- strain produced the highest 4VP titer obtained to date (187 g/L) with a high yield (90%). It is demonstrated for the first time that the producing strains taught herein are active in concentrated lignin solutions prepared from a solid lignin-rich corn stover residue when using an undecanol overlay or phase, and that 4VP can be produced from the CA present in those liquors. Compared to a previous report that employs hydrolysates generated by pretreating corn cobs containing only 20% lignin (Salgado et al., 2014), the method can release more CA and resulted in 10-fold higher 4VP titers and in 97% of the product extracted in the organic phase.

In some embodiments, the method has one or more of the following advantages: 1) The alkaline treatment of lignin-rich residues at high solids loading is an effective method to release high concentrations of coumarate; 2) undecanol was found to be an effective organic overlay or phase for 4VP extraction in the presence of microbes in concentrated solutions of the substrate coumarate; 3) C. glutamicum was found to perform better than other organisms in terms of 4VP production in the presence of high concentrations of lignin-derived monoaromatics, such as coumarate. These steps lead to a high conversion yield of 90% and an overlay or phase titer of 187 g/L in rich medium or 73% yield and 17 g/L titer in lignin liquors.

In some embodiments, the method produces 4VP, and further comprises: recovering the 4VP and polymerizing the 4VP into a polyhydroxystyrene, or derivative thereof. In some embodiments, the obtained product 4-vinylphenol can be subjected to chemical or enzymatic polymerization to generate polyhydroxystyrene derivatives that are used in the manufacture of soft plastics. The resulting high titers may help to reduce product purification costs and open the possibility to scale up the production and polymerization of 4VP. Since the enzymes remain active during the incubation period when using this method, the implementation of biological polymerization approaches by further genetic modification of the producing strains is feasible and worthy of being explored.

In some embodiments, the method offers the possibility to achieve high 4VP titers from high-substrate batch cultures with minimal intervention, in contrast to more expensive reactor configurations and substrate feeding profiles. The higher biocompatibility of undecanol towards the generated strains compared to previously reported solvents increases the number of organisms that can be employed for this and other related processes, which could diversify the range of bioproducts that can be generated. The use of undecanol also has the added benefit of enabling recovery of pure 4VP via a simple vacuum distillation due to the equal to more than about 40 ° C. difference in boiling points between 4VP (189-206° C.) and undecanol (243° C.).

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Cell density values measured during cultivation of the 9 strains in tryptic soy broth supplemented with 4VP or CA at different concentrations. Numbers in the horizontal axis indicate initial concentrations of 4VP or CA in grams per liter. TB refers to tryptic soy broth without any supplements. The 4VP concentrations were measured after 24 h of cultivation. The strains used are C. glutamicum (a), C. glutamicum phdA- (b), E. coli (c), C. glutamicum+PAD (d), C. glutamicum phdA-+PAD (e), E. coli+PAD (f), B. subtilis (g), B. subtilis+PAD (h), and B. amyloiquefaciens (1).

FIG. 2. 4VP yields (a, c) and concentrations (b, d) obtained in experiments using tryptic soy broth containing 25 g/L (a, b) or 75 g/L (c, d) of CA. Experiments were inoculated at an OD of 4 and yields were calculated based on the concentrations of 4VP in the aqueous and organic phase relative to the initial CA after 48 h of cultivation.

FIG. 3. Scheme of the microbial production of 4-vinylphenol (4VP) from p-coumaric acid (CA) in rich media or lignin-derived CA. PAD=phenolic acid decarboxylase.

FIG. 4. 4VP yields (a), concentrations (b), and cell densities (c) obtained when cultivating the producing strains in lignin liquors. Yields were calculated based on the concentrations of 4VP in the aqueous and organic phase relative to the initial CA after 48 h of cultivation.

FIG. 5. HPLC-UV chromatogram showing CA was the main compound detected.

### DETAILED DESCRIPTION OF THE INVENTION

Before the invention is described in detail, it is to be understood that, unless otherwise indicated, this invention is not limited to particular sequences, expression vectors, enzymes, host cells, microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

The terms “host cell” and “host microorganism” are used interchangeably herein to refer to a living biological cell, such as a microorganism, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given host cell; or (b) the structure or molecule may be naturally found in a given host cell, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host cell, wherein the expression vector contains a nucleic acid sequence coding for an enzyme that is not normally found in a host cell. With reference to the host cell's genome, then, the nucleic acid sequence that codes for the enzyme is heterologous.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host cell or cell. Only when the sequence of nucleic acids becomes stably replicated by the cell does the host cell or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host cell, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; intemucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

In some embodiments, the nucleotide sequences encoding the polypeptide are operatively linked to one or more promoters capable of transcription in the genetically modified host cell. In some embodiments, each nucleic acid of the one or more nucleic acids is a vector capable of stable introduction into and/or maintenance in the host cell. In some embodiments, the culturing step is under an anaerobic or microaerobic condition. In some embodiments, the genetically modified host cell is capable of producing one or more compounds in titers or yields equal to or more than the titers or yields described herein.

The PAD is any suitable PAD, such as any wild-type PAD, or any PAD with an amino acid sequence substantially identical to the amino acid sequences of SEQ ID NO: 1 or any wild-type PAD. The substantially identical PAD comprises one or more, or all, of the conserved residues identified herein.

The amino acid sequence of Bacillus amyloliquefaciens phenolic acid decarboxylase (PAD) (SEQ ID NO:1) is as follows:

MAYQDFREFL AALEKEGQLL TVNEEVKPEP DLGAAARAAS
NLGDKSPALL FNNIYGHHHA QVALNVIGSW PNHAMMLGMP
KDSPVKEQFF EFAKRYDAFP VPVKREETAP FHENEITEDI
NLFDILPLER INQGDGGYYL DKACVISRDL EDPDNFGKQN
VGIYRMQVKG KDRLGIQPVP QHDIAIHLRQ AEERGVNLPV
TIALGCEPVI TTAASTPLLY DQSEYEMAGA IQGEPYRIVK
SKLSDLDIPW GAEVVLEGEI LAGEREYEGP FGEFTGHYSG
GRSMPVIKIK RVYHRNNPIF EHLYLGMPWT ECDYMIGINT
CVPLYQQLKE AYPNEIVAVN AMYTHGLIAI ISTKTRYGGF
AKAVGMRALT TPHGLGYCKM VIVVDEDVDP FNLPQVMWAL
STKMHPKHDA VIIPDLSVLP LDPGSDPAGM THKMILDATT
PAPPETRGHY SQPLDSPLTT KEWEQKLMDL MNQ

In some embodiments, the PAD comprises one or more of the following conserved amino acid residues: binding site (Mn²⁺) at amino acid position 160, prenyl-FMN binding region at amino acid residue positions 160-165, prenyl-FMN binding region at amino acid residue positions 181-182, binding site (Mn²⁺) at amino acid position 182, binding site (Mn²⁺) at amino acid position 224, and/or proton donor active site at amino acid position 273.

In some embodiments, the PAD comprises one or more of the following conserved amino acid residues: N at amino acid position 160, NVGIYR (SEQ ID NO:2) at amino acid residue positions 160-165, QH at amino acid residue positions 181-182, H at amino acid position 182, E at amino acid position 224, and/or E at amino acid position 273.

### Enzymes, and Nucleic Acids Encoding Thereof

A homologous enzyme, or substantially identical enzyme, is an enzyme that has a polypeptide sequence that is at least 70%, 75%, 80%, 85%, 90%, 95% or 99% identical to any one of the enzymes described in this specification or in an incorporated reference. The homologous enzyme, or substantially identical enzyme, retains amino acids residues that are recognized as conserved for the enzyme. The homologous enzyme, or substantially identical enzyme, may have non-conserved amino acid residues replaced or found to be of a different amino acid, or amino acid(s) inserted or deleted, but which does not affect or has insignificant effect on the enzymatic activity of the homologous enzyme, or substantially identical enzyme. The homologous enzyme, or substantially identical enzyme, has an enzymatic activity that is identical or essentially identical to the enzymatic activity any one of the enzymes described in this specification or in an incorporated reference. The homologous enzyme, or substantially identical enzyme, may be found in nature or be an engineered mutant thereof.

The nucleic acid constructs of the present invention comprise nucleic acid sequences encoding one or more of the subject enzymes. The nucleic acid of the subject enzymes is operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xhol, Xmal, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host cell simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequencers) provides the ability to transfect host cell. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the isoprenol ensured. When added, any intermediate is present in an excess amount in the culture medium.

### Host Cells

The genetically modified host cell can be any prokaryotic or eukaryotic cell, with any genetic modifications, capable of express the polypeptide in accordance with the methods of the invention. In some embodiments, the host cell is naturally tolerant to CA and/or FA and to organic overlay or phase. Suitable eukaryotic host cells include, but are not limited to, fungal cells. Suitable fungal cells are yeast cells, such as yeast cells of the Saccharomyces genus. Generally, although not necessarily, the host cell is a yeast or a bacterium. Any prokaryotic or eukaryotic host cell may be used in the present method so long as it remains viable after being transformed with a sequence of nucleic acids. In some embodiments, the host cell is not adversely affected by the transduction of the necessary nucleic acid sequences, the subsequent expression of the proteins (i.e., enzymes), or the resulting intermediates required for carrying out the steps associated with the mevalonate pathway. For example, it is preferred that minimal “cross-talk” (i.e., interference) occur between the host cell's own metabolic processes and those processes involved with the mevalonate pathway.

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

The enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments, the host cell is Rhodosporidium toruloides or Pseudomonas putida. In some embodiments, the host cell is a Gram negative bacterium. In some embodiments, the host cell is of the phylum Proteobactera. In some embodiments, the host cell is of the class Gammaproteobacteria. In some embodiments, the host cell is of the order Enterobacteriales. In some embodiments, the host cell is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes.

Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtherias, Corynebacterium efficiens, Corynebacterium jeikeium, Corynebacterium macginleyi, Corynebacterium matruchotii, Corynebacterium minutissimum, Corynebacterium renals, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

In some embodiments, the host cell is B. subtilis RIK1285 strain. B. subtilis RIK1285 strain is commercially available from Takara Bio USA (San Jose, CA). In some embodiments, the host cell is C. glutamicum ATCC 13032 strain. C. glutamicum ATCC 13032 strain is commercially available from the American Type Culture Collection (Manassas, VA).

References cited herein are:

1. Barthelmebs L, Diviès C, Cavin J F. Expression in Escherichia coli of native and chimeric phenolic acic decarboxylases with modified enzymatic activities and method for screening recombinant E. coli strains expressing these enzymes. Appl Environ Microbiol. 2001;67:1063-9.

2. Beckham G T, Johnson C W, Karp E M, Salvachúa D, Varcon D R. Opportunities and challenges in biological lignin valorization. Curr Opin Biotechnol. 2016;42:40-53.

3. Blank L M, Ionidis G, Ebert B E, Bühler B, Schmid A. Metabolic response of Pseudomonas putida during redox biocatalysis in the presence of a second octanol phase. FEBS J. 2008;275:5173-90.

4. Bomble Y J, Lin C-Y, Amore A, Wei H, Holwerda E K, Ciesielski P N, et al. Lignocellulose deconstruction in the biosphere. Curr Opin Chem Biol. 2017;41:61-70.

5. Chen Y, Li L, Long L, Ding S. High cell-density cultivation of phenolic acid decarboxylase-expressing Escherichia coli and 4-vinylguaiacol bioproduction from ferulic acid by whole-cell catalysis. J Chem Technol Biotechnol. 2018;93:2415-21.

6. Cleto S, Jensen J V K, Wendisch V F, Lu T K. Corynebacterium glutamicum metabolic engineering with CRISPR interference (CRISPRi). ACS Synth Biol. 2016;5:375-85.

7. Fujiwara R, Noda S, Kawai Y, Tanaka T, Kondo A. 4-Vinylphenol procuction from glucose using recombinant Streptomyces mobaraense expressing a tyrosine ammonia lyase from Rhodobacter sphaeroides. Biotechnol Lett. 2016,38:1543-9.

8. Hu H, Li L, Ding S. An organic solvent-tolerant phenolic acic decarboxylase from Bacillus licheniformis for the efficient bioconversion of hydroxycinnamic acids to vinyl phenol derivatives. Appl Microbiol Biotechnol. 2015;99:5071-81.

9. Jäger W, Schäfer A, Pühler A, Labes G, Wohlleben W. Expression of the Bacillus subtilis sacB gene leads to sucrose sensitivity in the gram-positive bacterium Corynebacterium glutamicum but not in Streptomyces lividans. J BacterioL 1992;174:5462-5.

10. Jung D-H, Choi W, Choi K-Y, Jung E, Yun H, Kazlauskas R J, Kim B-G. Bio-conversion of p-coumaric acid to p-hydroxystyrene using phenolic add decarboxylase from B. amloliquefaciens in biphasic reaction system. Appl Microbiol Biotechnol. 2013;97:1501-11.

11. Kallscheuer N, Vogt M, Kappelmann J, Krumbach K, Noack S, Bott M, Marienhagen J. Identification of the phd gene cluster responsible or phenylpropanoid utilization in Corynebacterium glutamicum. Appl Microbiol Biotechnol. 2016;100:1871-81.

12. Licandro-Seraut H, Roussel C, Perpetuini G, Gervais P, Cavin J-F. Sensitivity to vinyl phenol derivatives produced by phenolic acid decarboxylase activity in Escherichia coli and several food-borne Gram-negative species. Appl Microbiol Biotechnol. 2013;97:7853-64.

13. Liebl W, Bayerl A, Schein B, Stillner U, Schleifer K H. High efficiency electroporation of intact Corynebacterium glutamicum cells. FEMS Microbiol Lett. 1989;65:299-303.

14. Liu Y, Nie Y, Lu X, Zhang X, He H, Pan F, et al. Cascade utilization of lignocellulosic biomass to high-value products. Green Chem. 2019,21:3499-535.

15. Liu Z-H, Le R K, Kosa M, Yang B, Yuan J, Ragauskas A J. Identifying and creating pathways to improve biological lignin valorization. Renew Sustain Energy Rev. 2019;105:349-62.

16. Li L, Long L, Ding S. Bioprocuction of high-concentration 4-vinylguaiacol using whole-cell catalysis harboring an organic solvent-tolerant phenolic acid decarboxylase from Bacillus atrophaeus. Front Microbiol. 2019;10:1798.

17. Li L, Wu X, Long L, Ding S. Functional autodisplay of phenolic acid decarboxylase using a GDSL autotransporter on escherichia coli for efficient catalysis of 4-hydroxycinnamic acids to vinylphenol derivatives. Catalysts 2019;9:634.

18. Parada-Fabián J C, Hernández-Sánchez H, Méndez-Tenorio A. Substrate specificity of the phenolic acic decarboxylase from Lactobacillus plantarum and related bacteria analyzed by molecular dynamics and dockin J Plant Biochem Biotechnol. 2018;28:1-14.

19. Plan T T P, Nguyen H D, Schumann W. Novel plasmid-based expression vectors for intra-and extracellular production of recombinant proteins in Bacillus subtilis. Protein Expr Purif. 2006;46:189-95.

20. Ralph J. Hydroxycinnamates in lignification. Phytochem Rev. 2010;9:65-83.

21. Ralph J, Lapierre C, Boerjan W. Lignin structure and its engineering. Curr Opin Biotechnol. 2019;56:240-9.

22. van der Rest M E, Lange C, Molenaar D. A heat shock following electroporation induces highly efficient transformation of Corynebacterium glutamicum with xenogeneic plasmid DNA. Appl Microbiol Biotechnol. 1999;52:541-5.

23. Rinaldi R, Jastrzebski R, Clough M T, Ralph J, Kennema M, Bruijnincx PCA, Weckhuysen B M. Paving the way for lignin valorisation: recent advances in bioengineering, biorefining and catalysis. Angew Chem Int Ed Engl. 2016;55:8164-215.

24. Rodriguez A, Salvachúa D, Katahira R, Black B A, Cleveland N S, Reed M, et al. Base-catalyzed depolymerization of solid lignin-rich streams enables microbial conversion. ACS Sustain Chem Eng. 2017;5:8171-80.

25. Salgado J M, Rodríguez-Solana R, Curiel J A, de Las Rivas B, Muñoz R, Domínquez J M. Bioproduction of 4-vinylphenol from corn cob alkaline hydrolyzate in two-phase extractive fermentation using free or immobilized recombinant E. coli expressing pad gene. Enzyme Microb Technol. 2014;58-59:22-8.

26. Sibout R, Le Bris P, Legée F, Cézard L, Renault H, Lapierre C. Structural redesigning arabidopsis lignins into alkali-soluble lignins through the expression of p-coumaroyl-CoA: monolignol transferase PMT. Plant Physiol. 2016;170:1358-66.

27. Verhoef S, Wierckx N, Westerhof R C M, de Winde J H, Ruijssenaars H J. Bioproduction of p-hydroxystyrene from glucose by the solvent-tolerant bacterium Pseudomonas putida S12 in a two-phase water-decanol fermentation. Appl Environ Microbiol. 2009;75:931-6.

28. Williamson J C, Bahrin N, Hardiman E M, Bugg T D H. Production of substituted styrene bioproducts from lignin and lignocellulose using engineered Pseudomonas putida KT2440. Biotechnol J. 2020;15:1900571.

29. Xu C, Arancon R A D, Labidi J, Luque R. Lignin depolymerisation strategies: towards valuable chemicals and fuels. Chem Soc Rev. 2014;43:7485-500.

30. Yoo C G, Meng X, Pu Y, Ragauskas A J. The critical role of lignin in lignocellulosic biomass conversion and recent pretreatment strategies: a comprehensive review. Bioresour Technol. 2020;301:122784.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### EXAMPLE 1

### Evaluation of Bacterial Hosts for Conversion of Lignin-Derived P-Coumaric Acid to 4-Vinylphenol

Hydroxycinnamic acids such as p-coumaric acid (CA) are chemically linked to lignin in grassy biomass with fairly labile ester bonds and therefore represent a straightforward opportunity to extract and valorize lignin components. In this work, we investigated the enzymatic conversion of CA extracted from lignocellulose to 4-vinylphenol (4VP) by expressing a microbial phenolic acid decarboxylase in Corynebacterium glutamicum, Escherichia coli, and Bacillus subtilis. The performance of the recombinant strains was evaluated in response to the substrate concentration in rich medium or a lignin liquor and the addition of an organic overlay to perform a continuous product extraction in batch cultures. We found that using undecanol as an overlay enhanced the 4VP titers under high substrate concentrations, while extracting >97% of the product from the aqueous phase. C. glutamicum showed the highest tolerance to CA and resulted in the accumulation of up to 187 g/L of 4VP from pure CA in the overlay with a 90% yield when using rich media, or 17 g/L of 4VP with a 73% yield from CA extracted from lignin. These results indicate that C. glutamicum is a suitable host for the high-level production of 4VP and that further bioprocess engineering strategies should be explored to optimize the production, extraction, and purification of 4VP from lignin with this organism.

Although some organisms, such as Pseudomonas putida and Streptomyces mobaraense, have been used for the production of 4VP from glucose [7, 27] or grass lignin feedstocks [28], the production of 4VP from CA has been attempted primarily in E. coli. Here we explored whether other bacterial species could be more tolerant to higher, more industrially relevant concentrations of substrate and product and to the presence of an organic overlay for product extraction. To do so, the PAD gene from Bacillus amyloliquefaciens was heterologously expressed in Corynebacterium glutamicum, Bacillus subtilis, and E. coli, and their 4VP production capabilities were determined using high-substrate batch cultivations. In an attempt to reduce the toxicity of the overlay, the performance of longer chain fatty alcohols, such as decanol and undecanol, was compared to the more commonly used overlay, octanol. Finally, liquors containing CA extracted from a lignin-rich fraction of lignocellulose were generated through an alkaline treatment and the strains were tested for 4VP production. The 4VP yields and titers obtained under these conditions were compared to those obtained from pure CA in rich medium.

### RESULTS AND DISCUSSION

### Initial Characterization of Strains

Three bacterial species were selected in this study for overexpression of a PAD enzyme: E. coli (Ec), B. subtilis (Bs) and C. glutamicum (Cg). Their ability to convert CA to 4VP was evaluated and compared to the species carrying the native gene, B. amyloliquefaciens (Ba). These organisms were chosen because they are common protein expression hosts with multiple engineering tools available and do not consume CA or 4VP, with the exception of Cg that is capable of assimilating CA. The metabolic pathway for assimilation of CA and other phenylpropanoids in C. glutamicum has been described in detail by Kallscheuer

et al., and interested readers are encouraged to consult that reference [11]. In an attempt to maximize CA conversion to 4VP in Cg, a clean deletion of phdA (coding for an acyl:CoA ligase required for CA degradation) was made to generate a strain unable to metabolize CA (Cg phdA-). It is also worth noting that wild-type Bs harbors a gene coding for a PAD enzyme in its genome; however, a plasmid-based expression is expected to have a positive effect on product formation rates and titers. The PAD from Ba was selected in this work to be overexpressed in all

organisms based on a previous study that identified it as having high specific activity [10]. However, the purpose of this work was not to study the performance of this particular enzyme but rather to determine if organisms such as Cg and Bs can be efficient 4VP producers.

The Ec, Cg and Cg phdA- strains were transformed with plasmids containing codon-optimized versions of the Ba PAD under the control of the strong IPTG-inducible promoter Ptac, called pJAM88 and pJAM89 (Table 1). These strains were named Ec PAD, Cg PAD and Cg phdA-PAD. Bs was transformed with a similar IPTG-inducible plasmid pJAM90 containing a

codon-optimized version of the Ba PAD gene controlled by the strong promoter Pgrac [19], resulting in strain Bs PAD.

TABLE 1
Strains and plasmids used in this work. The materials generated in this work
are stored at the Joint BioEnergy Institute (JBEI) and can be accessed at
webpage for public-registry.jbei.org with the registry ID numbers provided.

| Strain name | Genotype or description | Source | JBEI Registry ID |
|---|---|---|---|
| Cg | Corynebacterium glutamicum ATCC 13,032 | ATCC | |
| Cg ΔphdA | Corynebacterium glutamicum ΔphdA | This study | JPUB_018349 |
| Cg PAD | Corynebacterium glutamicum wild type with pJAM89 | This study | JPUB_018350 |
| Cg ΔphdA PAD | Corynebacterium glutamicum ΔphdA with pJAM89 | This study | JPUB_018352 |
| Bs | Bacillus subtilis RIK1285 (strain 168 derivative) | TakaraBio | |
| Bs PAD | Bacillus subtilis RIK1285 with pJAM90 | This study | JPUB_018354 |
| Ba | Bacillus amyloliquefaciens ATCC 23,350 | ATCC | |
| Ec | Escherichia coli DH5α | Zymo Research | |
| Ec PAD | Escherichia coli DH5α with pJAM88 | This study | JPUB_018346 |
| Plasmid name | Description | Source | JBEI Registry ID |
| pK18mobsacB | suicide vector | ATCC | |
| pZ8-Ptac | IPTG-inducible strong promoter expression vector | Addgene | |
| | | [6] | |
| pHT08 | IPTG-inducible strong promoter expression vector | MoBiTec | |
| pJAM88 | pZ8-Ptac with PAD codon-optimized for E. coli | This study | JPUB_018347 |
| pJAM89 | pZ8-Ptac with PAD codon-optimized for C. glutamicum | This study | JPUB_018351 |
| pJAM90 | pHT-08 with PAD codon-optimized for B. subtilis | This study | JPUB_018355 |

To test whether the PAD strains were able to convert CA to 4VP and evaluate their tolerance to different concentrations of substrate and product, growth experiments were performed in tryptic soy broth supplemented with 0.25 or 0.5 g/L of 4VP, or 1, 5 or 10 g/L of CA. This rich medium was used to provide all the organisms with glucose and amino acids for growth and cell maintenance and allow for continuous PAD protein production during the 4VP production process. The bacterial cell density was measured at the time of inoculation and after 24 h of incubation, and PAD expression was induced at the beginning of the cultivations.

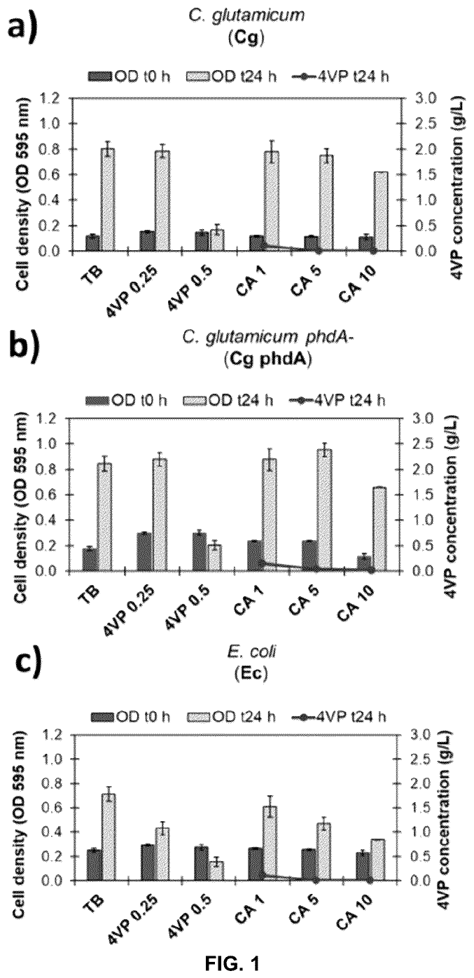

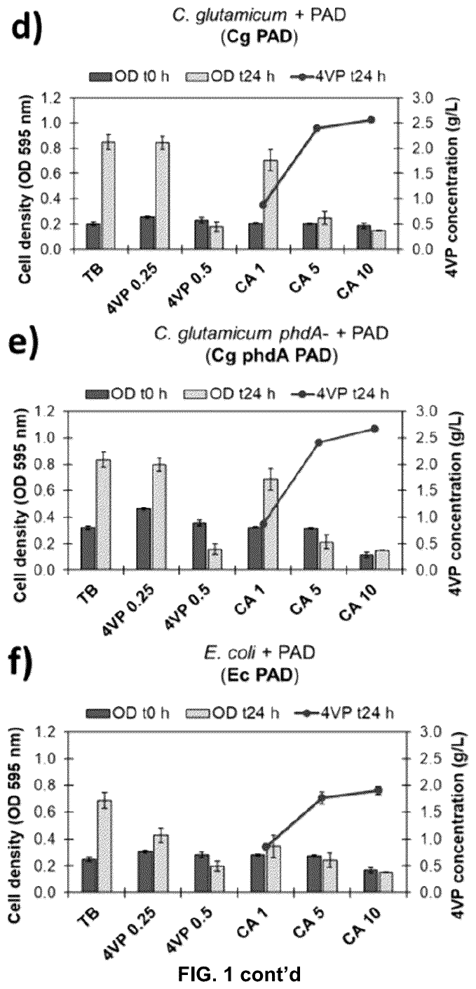

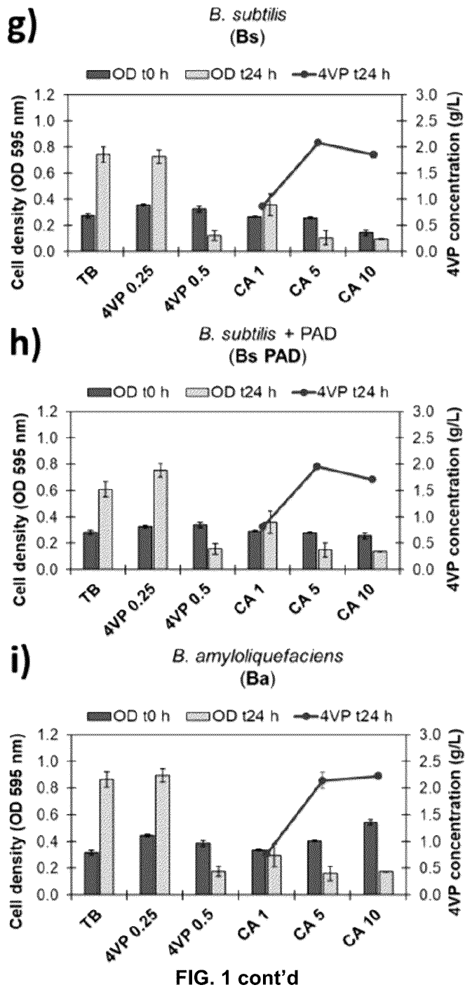

The results show that 4VP is very toxic to all tested organisms, although Ec appeared to be the most sensitive, displaying growth inhibition at concentrations as low as 0.25 g/L (FIG. 1c, f). This agrees with previously reported 4VP toxicity values for other E. coli strains [12]. Cg and Cg phdA- strains showed the highest tolerance to CA by being able to grow in the presence of up to 10 g/L of this compound (FIG. 1a, b), while all Bacillus species were unable to grow in media containing more than 1 g/L of CA (FIG. 1g-i).

Notably, the transformed Cg, Cg phdA-, and Ec cells exhibited increased sensitivity to CA compared to the non-transformed variants, suggesting that 4VP formation (and indirectly the initial CA concentration) is causing growth inhibition as a result of PAD expression. We confirmed that 4VP was produced by all strains that harbor a PAD in media containing CA (FIG. 1). As expected, Bs and Ba can naturally produce 4VP but not the untransformed Cg or Ec strains. Interestingly, Bs and Ba wild-type strains produced similar 4VP amounts as the transformed strains in this experiment, even when the cell density in the cultures did not increase. Product concentrations reached a plateau around 2.5 g/L despite increasing the initial CA concentration from 5 to 10 g/L. These observations indicate that even a small amount of PAD enzyme (cell densities as low as 0.1 OD) may be enough to reach inhibitory product concentrations and that 4VP toxicity (and not substrate availability) is likely limiting the product titers under these conditions.

### Use of an Organic Overlay and High Coumarate Concentrations

Previous reports have found that the microbial production of 4VP can be enhanced by using a biphasic cultivation system. One example is the extraction of 4VP from the aqueous phase with octanol in a continuous flow-bed reactor containing immobilized E. coli cells [10]. Although the partition coefficient for 4VP in a mixture of octanol and water is predicted to be high towards octanol, this solvent is also known to cause significant toxicity to microbes [3]. Therefore, we compared the performance of octanol to other mid-chain fatty alcohols with slightly longer alkane chains, decanol and undecanol, when added as overlays to the cultivation media (20% of the aqueous phase volume). To obtain high product concentrations and identify any limitations to the amount of 4VP that can be extracted with the overlays, the four recombinant strains were incubated in the presence of 25 or 75 g/L of CA.

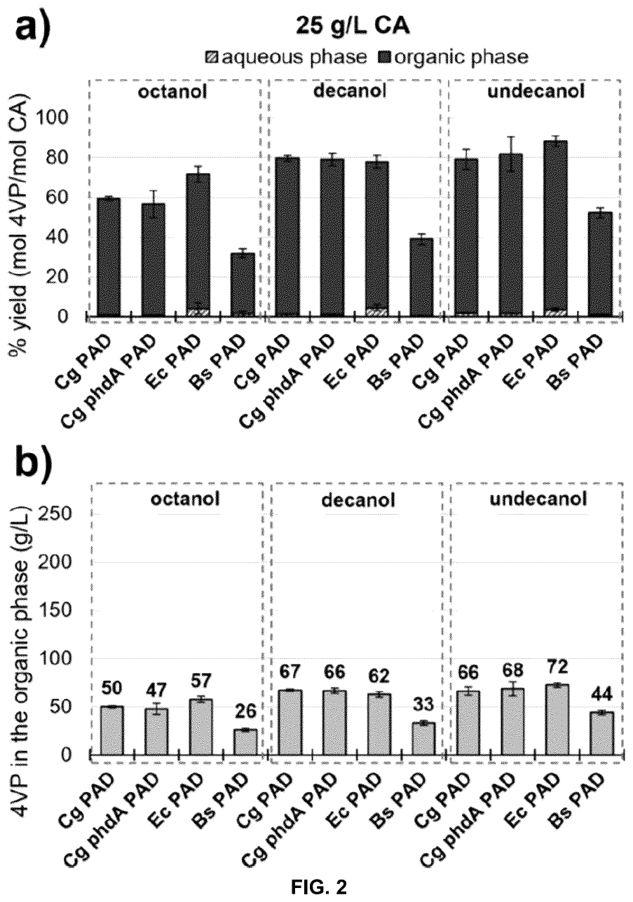

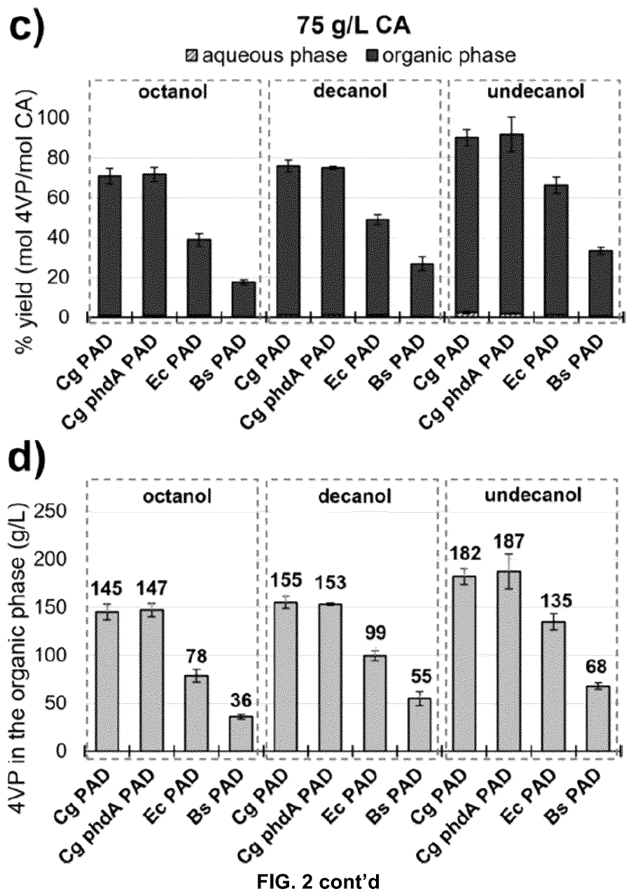

We observed differences in the 4VP yields and titers obtained with each overlay and organism. For example, Bs showed lower conversion yields and product titers than Cg or Ec (FIG. 2). We found that undecanol performs as well or better than octanol and decanol in terms of 4VP yields and titers, and this effect appears stronger when using a higher initial CA concentration. Although the titers and yields obtained with Cg and Ec in the presence of 25 g/L of CA were similar, Cg performed better in 75 g/L of CA, reaching a concentration of 4VP of 187 g/L in the undecanol overlay with a 90% conversion yield. This could be a consequence of the higher tolerance of this microorganism to CA, as discussed previously. Interestingly, no difference in 4VP production was found between Cg and Cg phdA-strains, which suggests that the CA assimilation pathway is not particularly active in the wild-type strain under these conditions.

Remarkably, over 97% of the 4VP produced was found in the organic phase, while more than 90% of the CA that was not consumed was found in the aqueous phase. This indicates that the three alcohols are able to efficiently extract and concentrate 4VP from the aqueous phase, while the substrate remains accessible to the cells. The use of an overlay markedly increased the production of 4VP when compared to monophasic cultures (FIG. 1). Undecanol was selected for further experiments based on the results shown in FIG. 2c, d and because it is the mid-chain fatty alcohol with the longest alkane chain that is liquid at room temperature, which could facilitate product recovery from the fermentation broth. It is important to consider that, besides helping cells to produce 4VP for a longer period of time, the inclusion of an overlay also helps to concentrate the product in a smaller volume and may be beneficial for a subsequent purification step.

### 4VP Production from Lignin-Extracted CA

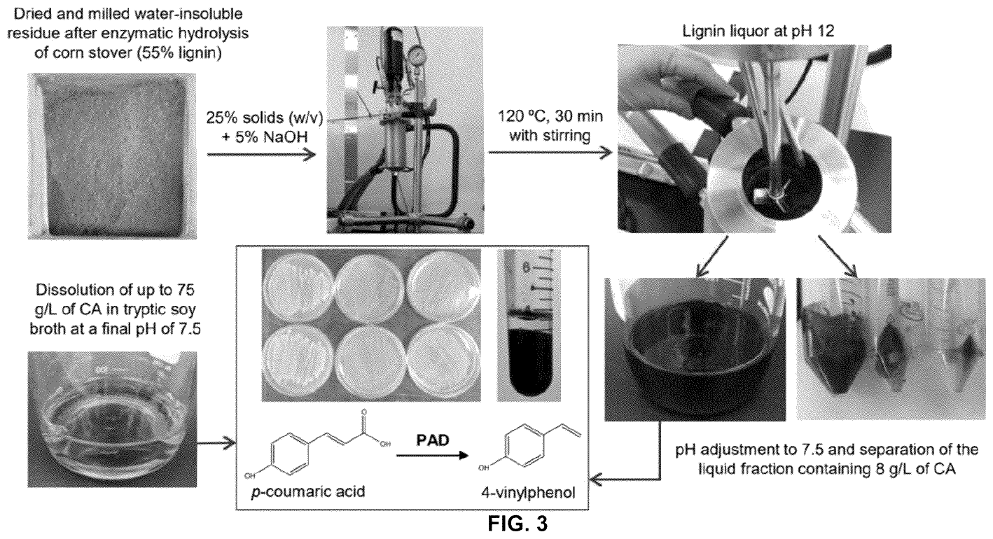

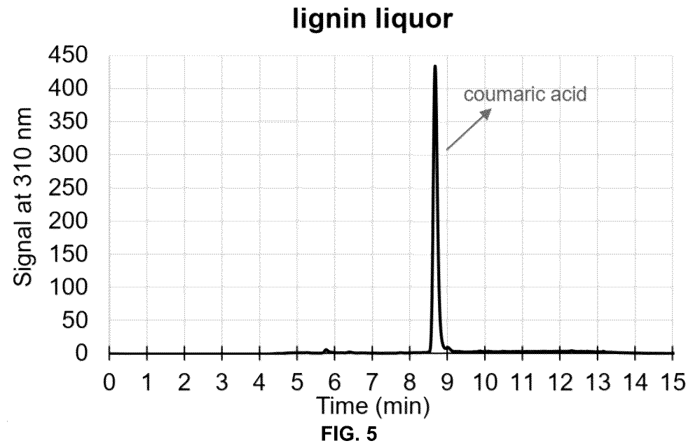

To explore a renewable source of 4VP production, the strains generated in this work were also tested using CA derived from lignocellulose. The lignin-rich substrate used in this study was derived from a dilute acid pretreatment and enzymatic saccharification of corn stover and is water insoluble. This substrate represents a typical solid waste stream that might be generated in a lignocellulosic biorefinery. To extract CA, the lignin-rich solid residue must be first solubilized under conditions that both promote the release of CA and generate a biocompatible liquor (i.e. having a pH and salt concentrations within the physiological range of the microbes and low amounts of toxic phenolic compounds). Previous reports indicate that CA yields close to 10 wt % of the total lignin-rich solids content can be obtained with a base-catalyzed depolymerization method using corn stover lignin [24]. Here, we applied a similar approach to prepare lignin liquors from the water-insoluble residue of corn stover (containing 55 wt % lignin), obtained after acid pretreatment and saccharification reactions in a pilot-scale process. A relatively high solids load of 25% (w/v) was used in the depolymerization reaction to maximize the CA concentration in solution (FIG. 3). The CA-rich liquor was pH-adjusted to 7.5 and a CA concentration of 8 g/L was measured. CA was the main compound detected in HPLC-UV chromatograms obtained from this material (FIG. 5).

The lignocellulose-derived CA was used to cultivate the 4VP producing strains. The cultivation setup used in the experiment shown in FIG. 2 included the use of multi-well plates and high initial cell densities. This was done to facilitate the screening of all combinations of strains, solvents, and substrate amounts, while minimizing the toxicity imposed by the high CA concentrations. However, this culture system is not amenable for repeated sampling of aqueous and organic phases to quantify metabolites or for monitoring cell growth in the presence of overlays and dark lignin liquors. Therefore, the incubations in CA-rich liquors were performed in culture tubes in presence of an undecanol overlay, and the OD, CA and 4VP concentrations were measured at 0, 12 and 24 h after inoculation. Considering that an organism able to produce 4VP at a high concentration and yield from lignin should not consume CA as a carbon source and may not necessarily be able to grow on lignin, high initial cell densities (OD ˜3.5) were also used in this experiment.

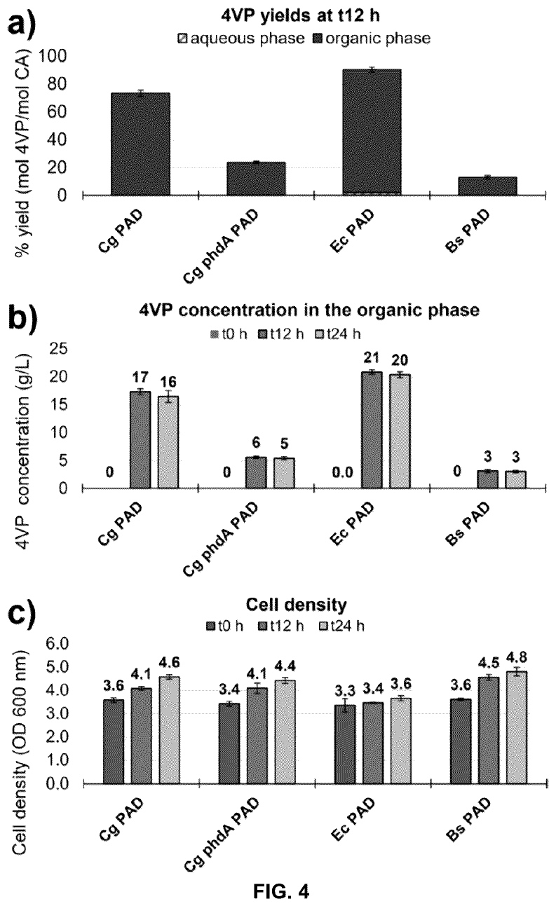

Maximum product concentrations were obtained within 12 h in all cases (FIG. 4). The Ec PAD strain showed the highest product concentration, reaching 21 g/L of 4VP with an 88% yield, followed by strain Cg PAD that produced 17 g/L with a 73% yield (FIG. 4). The Bs PAD strain was once again the lowest 4VP producer, accumulating only 3 g/L of 4VP. Interestingly, the Cg phdA-PAD strain resulted in low production values under these conditions (6 g/L of 4VP), suggesting that the absence of the acyl:CoA ligase could have adverse consequences for Cg when cultured in complex lignocellulose-derived media. Overall, the cell densities increased only slightly over the course of the cultivation but no correlation to the 4VP production values was found.

It is clear that the use of concentrated lignin liquors poses additional challenges to the bioconversion. Besides substrate, product and overlay tolerance, other factors such as the salt concentration and the presence of toxic lignin breakdown products could decrease the efficiency of the process. Therefore, the selection of an organism that can tolerate harsh media conditions and the development of appropriate feeding strategies are critical factors in lignin-derived bioprocesses. The approach described here resulted in an almost complete extraction of the product in the organic phase and higher 4VP concentrations than previously reported when using biological catalysts to produce 4VP from pure CA [8, 10, 16, 17] or lignocellulosic biomass [25].

### CONCLUSIONS

This work compared the performance of three organisms in a 4VP production process and demonstrates its production from CA extracted from a solid lignin-rich corn stover bioprocess residue. We found that the plasmid expression of a PAD enzyme in C. glutamicum strains resulted in the highest 4VP concentrations reported to date in any culture medium. In contrast, B. subtilis showed the lowest performance of the recombinant strains in all tested conditions. Continuous extraction of 4VP from the liquid phase with an organic overlay enabled high product concentrations and yields in batch mode, and the best results (187 g/L of 4VP from pure CA in the overlay with a 90% yield) were obtained when using undecanol. The use of undecanol also has the added benefit of enabling recovery of pure 4VP via a simple vacuum distillation due to the >40 ° C. difference in boiling points between 4VP (189-206° C.) and undecanol (243° C.). Our results showcase E. coli as the top performing organism in the lignin liquors, C. glutamicum as a promising new host for the high-level production of 4VP, and also indicate that the implementation of an extractive fermentation process could be beneficial to increase the conversion and recovery at higher scales. Overall, the combination of 4VP conversion host and extractive overlay described in his study outline a process that has a clear route to industrialization for this bioplastic monomer.

### METHODS

### Chemicals and Media

p-coumaric acid, dodecane, octanol, decanol, undecanol, diethyl ether, and all components of M9 medium were purchased from Sigma Aldrich. A 10% 4-vinylphenol solution in propylene glycol was purchased from Alfa Aesar. Tryptic Soy Broth was purchased from BD Difco™ (Franklin Lakes, NJ) and prepared according to the manufacturer's instructions. Coumaric acid (98%) powder was added to tryptic soy broth at 50° C. and solubilized by the slow addition of 10 N NaOH until the pH was approximately 7.5, then the medium was filter-sterilized using a 0.45 μm surfactant-free cellulose acetate membrane. M9 medium contained the following components (per liter): 6.78 g of Na₂HPO₄, 3 g of KH₂PO₄, 0.5 g of NaCl, 1.98 g of (NH₄)SO₄, 240 mg of MgSO₄, 12 mg of CaCl₂, 160 μg of CuSO₄, 160 μg of MnSO₄, 160 μg of FeSO4, 160 μg of ZnSO4, 20 mg of thiamine, and 20 mg of biotin. A tenfold concentrated M9 solution was used to supplement lignin liquors in a 1:9 v/v ratio to be used as cultivation media for production experiments.

E. coli and B. subtilis were maintained on LB (10 g/L tryptone, 5 g/L yeast extract, and 5 g/L NaCl) plates or liquid and C. glutamicum was maintained on LB agar/liquid or LBHIS agar plates (5 g/L tryptone, 5 g/L NaCl, 18.5 g/L brain heart infusion, 15 g agar, and 91 g/L sorbitol). C. glutamicum electroporation media are as follows; electrocompetent medium-1: autoclave 1 g tryptone, 0.5 g yeast extract, 1 g NaCl, 2.5 g LB media in 80 mL water and add filter sterilized 2.5 g glycine and 50 μL tween80, in 20 mL water; and BHIS: 18.5 g/L brain heart infusion and 91 g/L sorbitol [13].

### Plasmid Construction

The gene coding for a phenolic acid decarboxylase (PAD) enzyme in B. amyloliquefaciens was codon-optimized for B. subtilis, C. glutamicum, and E. coli, and synthesized by Genscript (Piscataway, NJ, USA). The B. subtilis PAD gene was then cloned into plasmid pHT08 to generate plasmid pJAM90 (Cm^{R}) via Gibson assembly using the DIVA platform at the Joint BioEnergy Institute. The codon-optimized genes for E. coli and C. glutamicum were cloned into the shuttle plasmid pZ8-Ptac to generate plasmids pJAM89 (Km^{R}) and pJAM88 (Km^{R}), respectively. All vectors allowed for the IPTG-inducible expression of the PAD enzyme.

To generate a clean deletion of phdA in C. glutamicum a deletion construct in a sacB suicide vector was made. The upstream and downstream regions of phdA (Cgl0284) were PCR amplified using C. glutamicum wild type genomic DNA with the following primers (sequences are shown in parentheses): KOphdA-F1-Eco (GTCgaattcCGATGACAGATGCACCCTGA) (SEQ ID NO:3), KOphdA-R1 (actagtgttcagtcgatcgttcgaaAGATCACGTTTGAAGTCA) (SEQ ID NO:4), KOphdA-F2 (ttcgaacgatcgactgaacactagtCACTTGGAGAACGCAATGA) (SEQ ID NO:5), and KOphdA-R2-HindIII (ATGaagcttGCGTCCGAAGAGGTCGGATA) (SEQ ID NO:6). The splice overlap PCR product, made by amplifying the upstream and downstream PCR product with primers KOphdA-F1-Eco and KOphdA-R2-HindIII, was ligated into zero blunt vector (Invitrogen). It was subsequently cut with EcoRI and HindIII and ligated into the similarly cut pK18mobsacB. The strains and plasmids used in this study are listed in Table 1 and those generated in this work can be accessed at webpage for public-registry.jbei.org using the part ID numbers provided.

### Generation of Strains

The microorganisms used in this work are described in Table 1. A C. glutamicum ATCC 13,032 strain that is unable to metabolize coumaric acid was generated by making a clean deletion of phdA, the gene coding for an acyl-CoA ligase (Cgl0284). This was accomplished by performing double homologous recombination and selection via the sacB system [9]. To integrate the deletion construct into C. glutamicum via transformation, wild type cells were made electrocompetent as per van der Rest with some variations [22]. C. glutamicum grown on LBHIS plates was inoculated into 10 mL LB 2% glucose and grown overnight at 200 rpm and 30° C. The overnight culture was inoculated into 1 L of electrocompetent medium-1 to an OD600nm of 0.3 and grown at 200 rpm at 18° C. until an OD600nm of 0.9-1.0. The cells were harvested, washed four times with ice cold 10% v/v glycerol, resuspended in 1 mL 10% v/v glycerol, and 150 μL aliquots were flash frozen in liquid nitrogen and stored at −80° C. until time of electroporation. The transformation to introduce the pdhA deletion construct was done by thawing the electrocompetent cells on ice and electroporating the cells in a 0.2 mm electroporation cuvette with 1 μg of plasmid using the conditions of 25 μF, 200 Ω, 3 kV, for 5 ms. Cells were resuspended in prewarmed (45° C.) 940 μL BHIS and incubated for 9 min at 45° C. The cells recovered at 200 rpm at 30° C. for 5 h before plating cells onto LBHIS agar supplemented with 30 μg/mL kanamycin and incubated at 30° C. The mutants were verified by PCR and by the inability to grow in CA as the sole carbon source.

In this study, strains with expression plasmids were generated by electroporation and briefly described here. E. coli DH5α Mix & Go competent cells were purchased from Zymo Research Corp. (Irvine, CA) and transformed by incubation with 1 μg of the plasmid DNA pJAM88 at room temperature for 5 min before plating on LB medium plates containing 30 μg/mL of kanamycin. B. subtilis RIK1285 cells were purchased from Takara Bio and made competent by harvesting them at the onset of stationary phase, following the instructions described by the supplier. Fresh competent cells were transformed with 2 μg of plasmid DNA pJAM90 and plated on LB plates with 10 μg/mL of chloramphenicol. C. glutamicum wild-type and the ΔphdA mutant were transformed by electroporation as above with these modifications. Both strains were made electrocompetent, thawed, and gently mixed with 100-200 ng of plasmid DNA (pJAM89), electroporated and allowed to recover for 2 h at 200 rpm at 30° C. before plating onto LBHIS with 30 μg/mL kanamycin. Transformants were verified by PCR and enzymatic digestion of the extracted plasmids.

### Microbial Cultivations

Cultivations were performed in 48-well flat bottom clear plates (Corning, USA) when an organic overlay was not added, in 48-well FlowerPlates (m2p, Germany) when an overlay was added to rich medium, and in 14 mL round-bottom plastic tubes for lignin liquors with an overlay. Seed cultures of all strains listed in Table 1 were started by inoculating cells from agar plates into tubes with 5 mL of tryptic soy broth and incubating at 30° C. with vigorous shaking for 12 h. For the experiments in the clear plates, inocula were prepared by mixing 1 mL of the seed cultures with 9 mL of fresh tryptic soy broth and incubated for 8 h. IPTG (0.5 mM final concentration) was added to the inocula after 6 h of incubation. Kanamycin (30 μg/mL) or chloramphenicol (10 μg/mL) were added as required for plasmid maintenance. To start the experiments in the clear plates, 15 μL of the inocula were combined with 485 μL of tryptic soy broth, with or without supplementation of 4VP or CA, and incubated with the plastic lid on in a shaking platform at 30° C. and 200 rpm. The optical density at 595 nm was measured using a DTX880 plate reader (Beckton-Coulter, USA).

For the experiments in the FlowerPlates or culture tubes, seed cultures were started in 5 mL of tryptic soy broth and incubating at 30° C. with vigorous shaking for 12 h with the appropriate antibiotics at the concentrations described above. Cells were transferred to 250 mL flasks containing 50 mL of tryptic soy broth (at a 1/10 dilution) and incubated at 30° C. with shaking in the presence of antibiotics. IPTG (0.5 mM final concentration) was added to the flasks after 6 h and incubated again for 2 h. Cell densities were measured, and the cells were centrifuged and resuspended in tryptic soy broth with CA or lignin liquors to obtain an initial OD of 4. IPTG (0.5 mM final concentration) was added at the beginning of the cultivations with plasmid-harboring strains. For cultivations in FlowerPlates, 800 μL of cells in tryptic soy broth media were combined with 200 μL of octanol, decanol or undecanol, covered with a gas-permeable sealing foil with a reduced evaporation layer (m2p, Germany), and incubated inside a humidity-controlled incubator shaker set at 30° C. and 900 rpm. After 48 h, the entire contents of each Flowerplate well were collected in 1.5 mL tubes and the dodecane layer, supernatant, and cells were separated by centrifugation. For cultivations in culture tubes, 3.2 mL of cells in lignin liquors were combined with 0.8 mL of undecanol and incubated at 30° C. and 200 rpm. Samples from the aqueous phase and undecanol phase were collected every 12 h. The cells in the aqueous phase were briefly centrifuged, resuspended in water, and transferred to a 96-well Costar black clear bottom plates (Corning, USA) to measure OD 600 nm using a SpectraMax m2 (Molecular Devices, USA). Each fraction was kept at −20° C. until analysis. All cultivations were performed in triplicate.

### Analytical Methods

CA and 4VP were quantified by HPLC in samples taken from the aqueous and organic phases of the cultivations or lignin liquors after diluting with water (aqueous samples) or pure acetonitrile (organic samples). The analysis was performed with an Agilent Technologies 1200 series instrument equipped with an Eclipse Plus Phenyl-hexyl column (250 mm length, 4.6 mm diameter, 5 μm particle size; Agilent Technologies, USA) kept at 50° C., and using an injection volume of 5 μl. The mobile phase was composed of 10 mM ammonium acetate in water (solvent A) and 10 mM ammonium acetate in acetonitrile 90% (solvent B), prepared from a stock solution of 100 mM ammonium acetate and 0.7% formic acid in water. The following mobile phase gradient profile was used: 30% B (0 min; 0.5 mL/min), 80% B (12 min; 0.5 mL/min), 100% B (12.1 min; 0.5 mL/min), 100% B (12.6 min; 1 mL/min), 30% B (12.8 min; 1 mL/min), 30% B (15.6 min; 1 mL/min). A Poroshell 120 EC-C18 column (50 mm length, 3 mm diameter, 2.7 μm particle size; Agilent Technologies, USA) kept at 22° C. was also employed for high-throughput analysis of samples collected from FlowerPlate cultivations, by using 40% (v/v) acetonitrile in water and 0.04% formic acid as mobile phase. Coumaric acid and 4-vinylphenol were quantified with a UV detector using 310 nm and 254 nm wavelengths, respectively. Analyte concentrations were calculated by comparison of the resulting peak areas to calibration curves made with commercial compounds.

### Lignin Preparation, Depolymerization and Coumaric Acid Extraction

Corn stover was pretreated in a horizontal screw reactor at 175° C. with 30 g H₂SO₄/kg dry biomass, 30% (w/w) total solids loading, and a residence time of 8 min (Shekiro III). The pretreated corn stover was then neutralized with 40% (w/w) NaOH to pH 5.5 before loading of the enzymes. Enzymatic saccharification was performed in a 50 L IKA SPP50 reactor with 15% (w/w) solid loading. The total batch mass was 30 kg with cellulose (CTec2) 64 mg protein/g dry biomass and xylanase (HTec2) 8 mg protein/g dry biomass. After 96 h incubation, the reaction was stopped, and the slurry was transferred out. Lignin rich solid was separated from the hydrolysate using a basket centrifuge with a polypropylene filter bag (pore size 25-30 micron) as the liner.

The lignin-rich residue obtained after saccharification was washed with water to remove low molecular weight compounds. This material was oven-dried at 50° C. for 48 h, ground with a mortar and pestle and milled using a 2 mm sieve. Compositional analysis of the dried sample revealed lignin, glucan, and xylan contents of 54.5%, 13.7%, and 3.8%, respectively. This sample was subjected to a base-catalyzed depolymerization process, as follows: (1) 37.5 g of solids were mixed with 150 mL of 5% NaOH in a stainless steel pressure reactor containing an impeller and a jacket for temperature control (Buchiglas, Switzerland); (2) a reaction was carried at 120° C. and for 30 min (in addition to a 35 min heating up and a 25 min heating down ramps); (3) a lignin liquor with a pH of 12 was recovered and the pH was adjusted to 7.5 with concentrated H₂So₄; (4) the liquid phase was recovered by centrifugation and filtered through 0.45 μm surfactant-free cellulose acetate membranes. The recovered liquor was supplemented with M9 salts ( 1/10 vol. of a 10×M9 stock), resulting in a concentration of 8 g/L of CA.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A method for producing a 4-vinylphenol (4VP) and/or 4-vinylguaiacol (4VG), the method comprising: (a) providing a host cell capable of expressing a polypeptide having a phenolic acid decarboxylase (PAD) enzymatic activity wherein the polypeptide is capable of converting p-coumaric (CA) and/or ferulic acid (FA) into 4-vinylphenol (4VP) and/or 4-vinylguaiacol (4VG), respectively; and (b) culturing the host cell in a culture medium to express the polypeptide such that the polypeptide converts CA and/or FA into 4VP and/or 4VG, respectively; wherein the culture medium comprises an organic overlay or phase.

2. The method of claim 1, the method further comprising: (c) recovering the 4VP and/or 4VG.

3. The method of claim 2, wherein organic overlay or phase comprises a saturated or unsaturated fatty alcohol, and the recovering step comprises distilling to separate the 4VP and/or 4VG from the saturated or unsaturated fatty alcohol.

4. The method of claim 3, wherein the difference between the boiling point of 4VP or 4VG and the boiling point of the saturated or unsaturated fatty alcohol is equal to or more than about 10° C.

5. The method of claim 1, wherein the host cell is capable of growth on pretreated lignocellulosic hydrolysate as a carbon source, and the culture medium comprises pretreated lignocellulosic hydrolysate.

6. The method of claim 1, wherein the host cell is deleted or knocked out for expression of endogenous pta, poxB, and/or ackA genes.

7. The method of claim 6, wherein the host cell is modified to have an increased expression of an acetyl-CoA synthetase (ACS).

8. The method of claim 1, wherein the polypeptide comprises: (i) an amino acid sequence having at least about 70% amino acid sequence identity with SEQ ID NO:1; and, (ii) one or more of the following conserved amino acid residues: N at amino acid position 160, SEQ ID NO:2 at amino acid residue positions 160-165, QH at amino acid residue positions 181-182, H at amino acid position 182, E at amino acid position 224, and/or E at amino acid position 273.

9. The method of claim 1, wherein the organic overlay or phase comprises a saturated or unsaturated fatty alcohol as a mechanism for sequestering the 4VP or 4VG product from the aqueous solution.

10. The method of claim 9, wherein the organic overlay or phase comprises a saturated fatty alcohol having a chemical structure: R″—OH, wherein R″ is a C₁ to C₂₀ alkyl.

11. The method of claim 10, wherein R″ is an alkyl with 1, 2, 3, 4, 5, 6, 7, 8, 9, 10, 11, 12, 13, 14, 15, 16, 17, 18, 19, or 20 carbon atoms, or having a range of any two preceding values thereof.

12. The method of claim 9, wherein the organic overlay or phase comprises an unsaturated fatty alcohol the organic overlay or phase comprises a chemical structure: α—CR═CR′—β—OH, wherein α and β are each independently a C₁ to C₂₀ alkyl, and R, and R′ are each independently H or a C₁ to C₂₀ alkyl, wherein 1, 2, 3, or 4 of α, β, R, and R′ comprises one or more hydroxyl groups.

13. The method of claim 12, wherein α and β are each independently a C₅ to C₂₀ alkyl, and R and R′ are each independently H or a C₅ to C₂₀ alkyl.

14. The method of claim 13, wherein the unsaturated fatty alcohol has the following chemical structure:

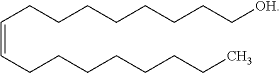 [SMILES: CCCCCCCC/C=C\CCCCCCCCO]

15. The method of claim 9, wherein the saturated or unsaturated fatty alcohol is a liquid at about room temperature, or about 20° C., 25° C., 30° C., 35° C., or 40 ° C.

16. The method of claim 9, wherein the saturated or unsaturated fatty alcohol is not toxic to the host cell, and/or does not inhibit the enzymatic activity of PAD.

17. The method of claim 1, wherein the method results in the 4VP and/or 4VG extracted and concentrated in the organic phase reaches a titer equal to or more than about 100 g/L, 110 g/L, 120 g/L, 130 g/L, 140 g/L, 150 g/L, 160 g/L, 170 g/L, 180 g/L, or 190 g/L, or a range between any two preceding values.

18. The method of claim 1, wherein the CA and/or FA are derived or obtained from a biomass.

19. A genetically modified host cell capable of producing a 4-vinylphenol (4VP) and/or 4-vinylguaiacol (4VG) comprising: a polypeptide having a phenolic acid decarboxylase (PAD) enzymatic activity wherein the polypeptide is capable of converting p-coumaric (CA) and/or ferulic acid (FA) into 4-vinylphenol (4VP) and/or 4-vinylguaiacol (4VG), comprising: (i) an amino acid sequence having at least about 70% amino acid sequence identity with SEQ ID NO:1; and, (ii) one or more of the following conserved amino acid residues: N at amino acid position 160, SEQ ID NO:2 at amino acid residue positions 160-165, QH at amino acid residue positions 181-182, H at amino acid position 182, E at amino acid position 224, and/or E at amino acid position 273; wherein the host cell is capable of growth on pretreated lignocellulosic hydrolysate as a carbon source, and the host cell is deleted or knocked out for expression of endogenous pta, poxB, and/or ackA genes.

20. The genetically modified host cell of claim 19, wherein the host cell is modified to have an increased expression of an acetyl-CoA synthetase (ACS).


SEQUENCE LISTING (6 sequences)

SEQ ID NO: 1 - protein, 473 aa, Bacillus amyloliquefaciens
MAYQDFREFLAALEKEGQLLTVNEEVKPEPDLGAAARAASNLGDKSPALLFNNIYGHHHAQVALNVIGSWPNHAMMLGMPKDSPVKEQFFEFAKRYDAFPVPVKREETAPFHENEITEDINLFDILPLFRINQGDGGYYLDKACVISRDLEDPDNFGKQNVGIYRMQVKGKDRLGIQPVPQHDIAIHLRQAEERGVNLPVTIALGCEPVITTAASTPLLYDQSEYEMAGAIQGEPYRIVKSKLSDLDIPWGAEVVLEGEILAGEREYEGPFGEFTGHYSGGRSMPVIKIKRVYHRNNPIFEHLYLGMPWTECDYMIGINTCVPLYQQLKEAYPNEIVAVNAMYTHGLIAIISTKTRYGGFAKAVGMRALTTPHGLGYCKMVIVVDEDVDPFNLPQVMWALSTKMHPKHDAVIIPDLSVLPLDPGSDPAGMTHKMILDATTPAPPETRGHYSQPLDSPLTTKEWEQKLMDLMNQ

SEQ ID NO: 2 - protein, 6 aa, Bacillus amyloliquefaciens
NVGIYR

SEQ ID NO: 3 - DNA, 29 bp, synthetic construct
  source 1..29: mol_type = other DNA
gtcgaattccgatgacagatgcaccctga

SEQ ID NO: 4 - DNA, 43 bp, synthetic construct
  source 1..43: mol_type = other DNA
actagtgttcagtcgatcgttcgaaagatcacgtttgaagtca

SEQ ID NO: 5 - DNA, 44 bp, synthetic construct
  source 1..44: mol_type = other DNA
ttcgaacgatcgactgaacactagtcacttggagaacgcaatga

SEQ ID NO: 6 - DNA, 29 bp, synthetic construct
  source 1..29: mol_type = other DNA
atgaagcttgcgtccgaagaggtcggata